(19) United States
(12) Patent Application Publication
(10) Pub. No.: US 2010/0191190 A1
(43) Pub. Date: Jul. 29, 2010

(54) BODY LINE MANAGEMENT SYSTEM

(76) Inventors: Charles E. Nokes, JR., Vancouver, WA (US); Valerie Lynn Vance-Talbert, Vancouver, WA (US)

Correspondence Address: CHERNOFF, VILHAUER, MCCLUNG & STENZEL, LLP, 601 SW Second Avenue, Suite 1600, PORTLAND, OR, 97204-3157, US

(21) Appl. No.: 12/362,404

(22) Filed: Jan. 29, 2009

(51) Int. Cl. A61M 25/02

(52) U.S. Cl. 604/174

(57) ABSTRACT

A body line management system, including methods and apparatuses for a patient dressing which includes retention wheels, about which IV, catheter, monitoring lines, biofeedback lines, or similar lines can be wrapped to secure the lines from unintentional disruption or dislodgment while permitting a practitioner to check the line as necessary.

### CROSS-REFERENCE TO RELATED APPLICATIONS

None

### BACKGROUND OF THE INVENTION

Patients with both minor and major illness and injury are often faced with using for some period of time a variety of body lines, such as a central venous access devices (CVAD), PICC Lines, IV's, feeding tubes, elimination tubes, chest tubes, arterial lines, mechanical ventilator tubing, drain tubes and all other catheters, as well as electronic, EKG and other type of bio sensor wiring.

Many patients are anxious about maintaining these body lines. Disruption of body lines can be painful for the patient, traumatizing the surrounding tissue and frequently requiring medical practitioners to adjust or replace the line.

Body line disruption may also increase the risk for dangerous infections and complications, which can lead to prolonged hospitalization or even death.

It is critically important to maintain all body lines with an eye towards preventing infection. Even greater watchfulness is warranted when caring for patients who are at increased risk of developing a catheter-related bloodstream infection, including: immunocompromised patients (e.g., oncology patients, HIV+ patients, those receiving long-term steroids), patients with other infections, those with multi-lumen CVADs, and those receiving parenteral nutrition.

Line-related infections occur in several different ways: contamination of the device by skin flora on insertion; migration down the cannula tract from the skin; contamination through the hub during manipulation; and seeding from another site of infection. Rarely, a contaminated infusate may be the culprit.

Practitioners need to assess the insertion site for many symptoms, including drainage, edema, and color or temperature changes, but such assessment is made more difficult when the patient has suffered unintentional line disruptions and is fearful of further painful disruption caused by removing adhesive dressings.

Conventional means of minimizing the risk of body line-related infections include proper hand washing by healthcare personnel, using maximal sterile barriers at the time of insertion, use of chlorhexidine gluconate (CHG) based skin preparations for insertion and care, careful site insertion selection, and frequent inspection to review whether CVADs are still necessary and removing them as soon as they are no longer necessary. For additional protection against line infections, some facilities use CHG-impregnated sponges at the catheter exit site.

Known in the art is the use of a manufactured catheter stabilization device specifically engineered to prevent catheter movement into or out of the insertion site. If CVADs are not sutured in place (a practice associated with additional sources of infection), some method of stabilization other than the dressing must be employed.

To date, known stabilization methods have only addressed the point of entry, and have done little if anything to address destabilization which line disruption may cause. These methods include use of a manufactured catheter stabilization device which may contain an adhesive anchoring pad to help reduce catheter dislodgment and the need for removal and reinsertion. Other known methods include use of sterile tape and surgical strips, sutures, and other dressings. These known methods alone fail to provide line stabilization at any point in the line other than at the point of insertion. In fact, because sutures break the skin, they increase the potential for irritation and infection. The Infusion Nursing Standards of Practice no longer list dressings as stabilization devices. Although dressings protect the insertion site and skin, practitioners doubt that known dressings enhance catheter stabilization. Gauze dressings preclude viewing the insertion site so must be removed. When removing a dressing, the practitioner risks accidentally dislodging the line.

Frequently a patient must have a line in place for an extended period of time, when the patient may be further compromised by diminished mobility and coordination. Pediatric patients and patients with cognitive difficulties will be even less likely to understand the need to protect the line and have to be physically restrained to prevent line disruption. Patients in transport, particularly emergency transport, run a significant risk of line disruption.

There is a need for a line management system which protects a line from disruption and enhances its stabilization, whether used in conjunction with known stabilization methods or not.

There is a need for a line management system which allows easy assessment of a line insertion site easily without altering the flow of fluid through the line.

There is a need for a line management system which is convenient, durable, and easy to use, allowing practitioners to incorporate the system into their standard practices for best patient care.

### BRIEF SUMMARY OF THE INVENTION

Described is a line management system which protects a line from disruption and enhances its stabilization, which permits use in conjunction with known stabilization methods.

Described is a line management system which allows easy assessment of a line insertion site easily without compromising the integrity of the line.

Described is a line management system which is convenient, durable, and easy to use, allowing practitioners to incorporate the system into their standard practices for best patient care.

The foregoing and other objectives, features, and advantages of the invention will be more readily understood upon consideration of the following detailed description of the invention, taken in conjunction with the accompanying drawings.

### BRIEF DESCRIPTION OF THE SEVERAL VIEWS OF THE DRAWINGS

FIG. 1 is a plan drawing of a preferred embodiment of the system as utilized in sleeve dressing.

FIG. 2 is a plan drawing of a preferred embodiment of the system as utilized in central line bib dressing.

FIG. 3a is a front perspective drawing of a preferred embodiment of the system as utilized in a full care central line vest.

FIG. 3b is a front perspective drawing of a preferred embodiment of the system as utilized in a full care central line vest.

FIG. 3c is a back perspective drawing of a preferred embodiment of the system as utilized in a full care central line vest.

FIG. 4 is a plan drawing of a preferred embodiment of the system as utilized in an infusion sleeve dressing.

FIG. 5 is a plan drawing of a preferred embodiment of the system as utilized in a neonatal line sleeve dressing.

FIG. 6 is plan drawing of a preferred embodiment of the system as utilized in a line management lanyard.

FIG. 7 is a plan drawing of a preferred embodiment of the system as utilized in an electro line management clip.

FIG. 8 is a plan drawing of a preferred embodiment of the system as utilized in an arm line cuff dressing.

FIG. 9 is a plan drawing of a preferred embodiment of the system as utilized in drain bag pouch dressing.

FIG. 10 is a plan drawing of a preferred embodiment of the system as utilized in leg line cuff dressing.

### DETAILED DESCRIPTION OF PREFERRED EMBODIMENT

The system described herein has been entitled a “Body Line Management System” to emphasize its utility with all manner of apparatuses which come in close contact with a patent and are monitored by or utilize a line of some type for transport. Such apparatuses include but are not limited to those used to transport fluids to a patient, or used to transport regulatory signals to a patent, or used to transport fluids away from a patent, or used to transmit information from a patent to a monitoring device.

Use of the system involves providing a dressing for a patient. The dressing includes at least one retention wheel, but a plurality of retention wheels in a series. As shown, the dressing is secured to a patent. A body line is inserted into a patent at a point of entry. Use of the system further involves wrapping at least a portion of a body line around the retention wheel or wheels. The center of a retention wheel is generally fixed to the dressing. The edges of the retention wheels are removably fixed to the dressing. At the time of wrapping, the edges of the retention wheels are not fixed, but after wrapping, the edges should be fixed to assist in retaining the body line wrapping. The edges of the retention wheels are preferably removably affixed with Velcro or similar product. Use of this system secures and stabilizes the body line, while allowing the non-wrapped portion of the body line to traverse the distance between the patent and the apparatus.

Retention wheels may be of a size to accommodate a single body line, or of a size to accommodate larger or multiple body lines. The fixed center of a retention wheel is of a size to prevent crimping of a body line when wrapped.

Use of this system further involves securing the dressing to the patent. In the preferred embodiments, the dressings are designed to be removably secured with Velcro or similar material. The dressings could also be secured with other removable attachment means such as ties, buckles, clips, or retention devices.

Use of this system may further involve providing additional retention straps to assist in securing the body line.

In the preferred embodiment, the dressing is largely made of a lightweight FDA approved material. To preserve sanitary conditions, the dressing may have an indicator to alert a practitioner when a dressing has been previously used.

The dressing is also preferably secured in place through use of straps which are removably affixed to the dressing with Velcro or similar product which is easy to use and permits adjustments if needed. The dressing could alternately be affixed with tape or buckles or other fixing means.

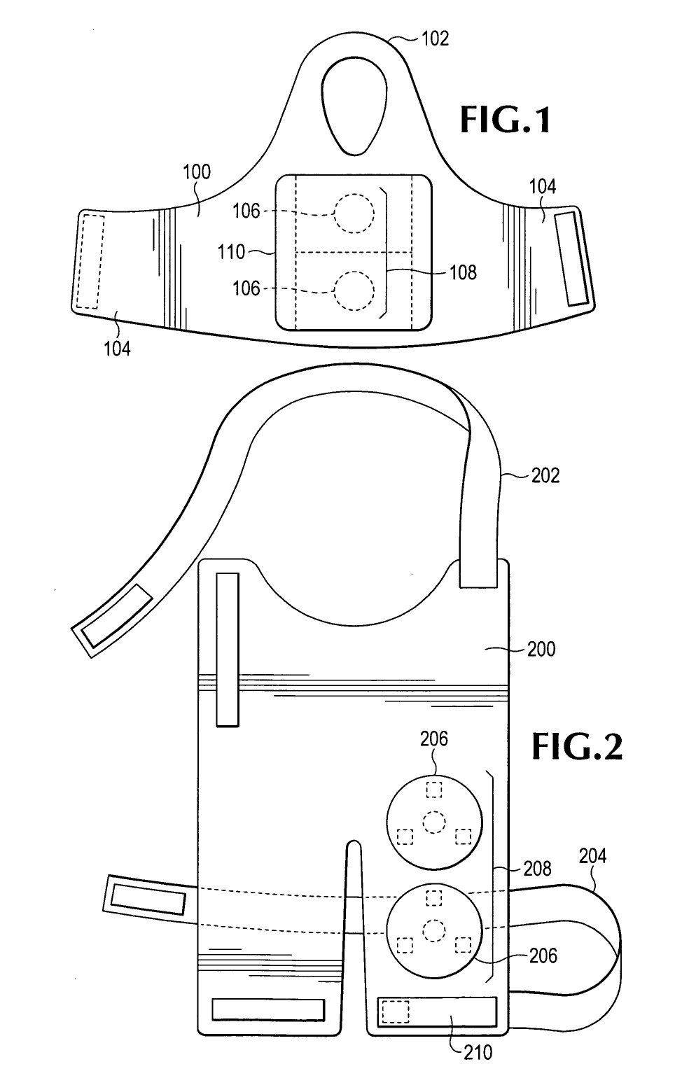

Use of this system in a preferred embodiment is shown in FIG. 1. In this embodiment, dressing takes the form of a sleeve 100 which has a thumb loop 102 which fits over a patient's thumb while arm strap 104 is secured around the patient's wrist or arm. At least one retention wheels 106 is affixed to the sleeve. As shown, two retention wheels 106 are preferable, and the wheels 106 are arranged in an array 108 which permits a practitioner to wrap a body line [not shown] around the wheels 106 in a manner which does not crimp the line but takes up any slack in the line which could be hazardous to the patent if left loose. Once the line is wrapped around the wheels 106, a cover 110 is secured over the array 108. The cover 110 is preferably provided affixed to the sleeve on at least one side of the cover 110, and removably affixed on at least on other side of the cover 110, to permit a practitioner to detach one side of the cover, check the line and then replace the cover if necessary.

Use of this system in an alternate embodiment is shown in FIG. 2. In this embodiment, the dressing takes the form of a bib 200 which fits over a patent's chest and is secured with an adjustable neck strap 202 and waist strap 204. The bib as shown has two retention wheels 206 in an array 208 which are larger in size to accommodate multiple body lines if necessary. The body line may be additionally secured with a line strap 210. The bib is well suited for central lines and body lines in a patent's upper arm, neck, and torso.

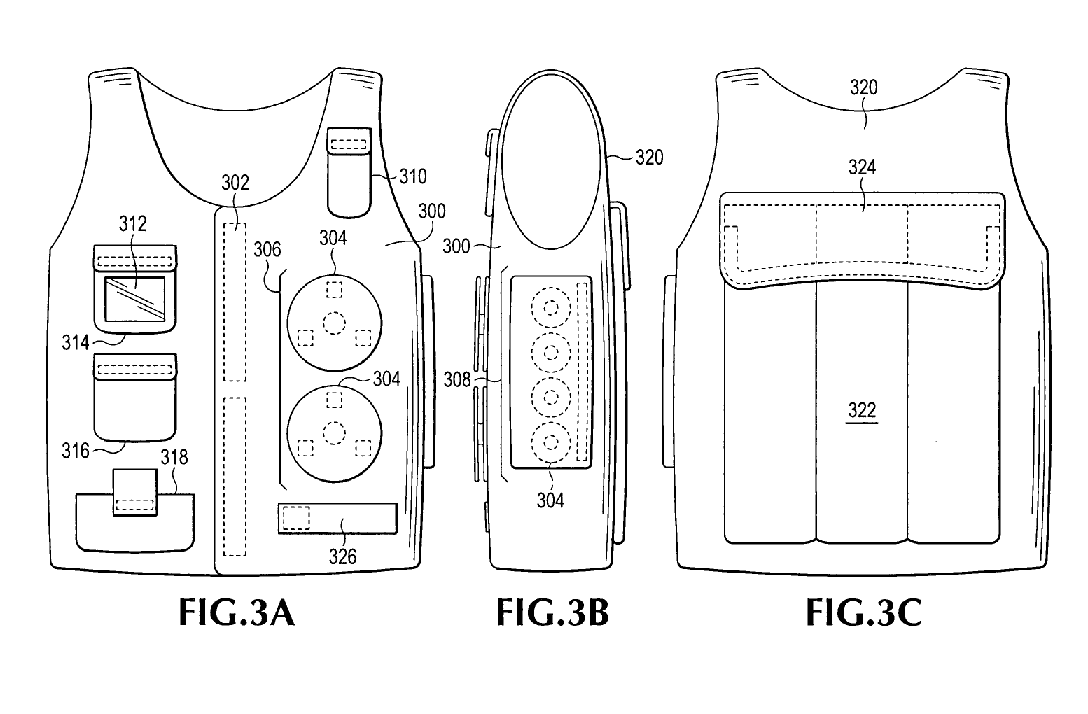

FIG. 3a shows an alternate preferred embodiment. In this embodiment, the dressing take the form a vest 300 which is secured around a patent's chest and has a central adjustable closure 302. The vest as shown in FIG. 3a and 3b has two retention wheels 304 in a front series 306, and four retention wheels 304 in a side series 308. The side series 308 is preferably used for monitoring lines such as those for monitoring EKGs, blood oxygen, and pulse. The front series 306 is preferably used for central lines and body lines in a patent's upper arm, neck, and torso. The vest as shown is equipped with a radio frequency transmitting device pocket 310, a monitor pocket 312 with clear cover 314, an accessory pocket 316, and a pump pocket 318. The vest may be manufactured with some or all of these pockets included in various convenient locations. The body line may be additionally secured with a line strap 326.

FIG. 3c shows the back 320 of the vest 300. The back 320 may include back pockets 322 which are protected from compression by a turtle shell (not shown). The pockets 322 may preferably used to hold IV fluid bags, drain bags, pumps, and battery packs. The pockets 322 have a closure 324 to secure their contents. The turtle shell is preferably a semi-rigid pad inserted into a back pocket 322. The turtle shell permits the patient to sit and lie back without disturbing function and while providing some comfort.

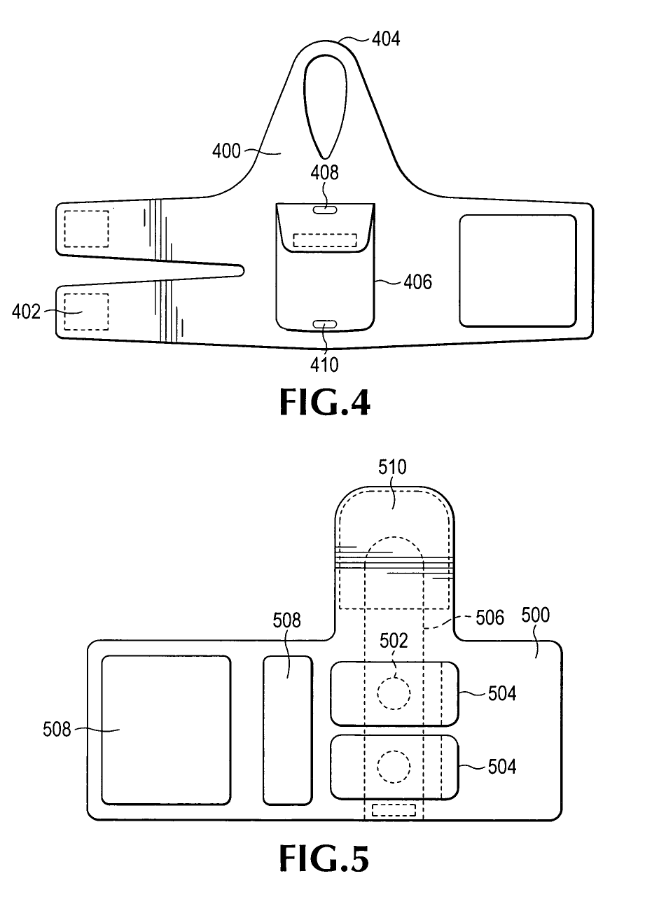

Another alternate embodiment showing use of this system is seen in FIG. 4. In this embodiment, the dressing take the form a infusion sleeve 400 which is secured around a patent's arm with an adjustable closure 402 and additionally anchored with a thumb loop 404. The sleeve 400 has a pocket 406 which holds an enFlow or similar fluid warming or filtering device [not shown]. The pocket 406 is equipped with a input slot 408 and discharge slot 410 to aid in line management when the pocket cover 412 is secured.

Another alternate embodiment showing use of this system is seen in FIG. 5. In this embodiment, the dressing take the form a neonatal sleeve 500. The neonatal sleeve 500 has two retention wheels 502 in series. The retention wheels may be covered with a fixably removable cover 504 which allows for easy adjustment of the body lines. Additionally, the sleeve 500 has a stiffener pocket 506 to permit insertion of a stiffening agent [not shown] such as a tongue depressor to aid in immobilization of the patient's joints if needed. The neonatal sleeve 500 is secured to the patent by means of a fixably removable strap 508 such as Velcro or similar product and a mitt 510. The patient's hand or foot maybe inserted into the mitt 510 while the strap 508 is secured around the patent's arm or leg as appropriate.

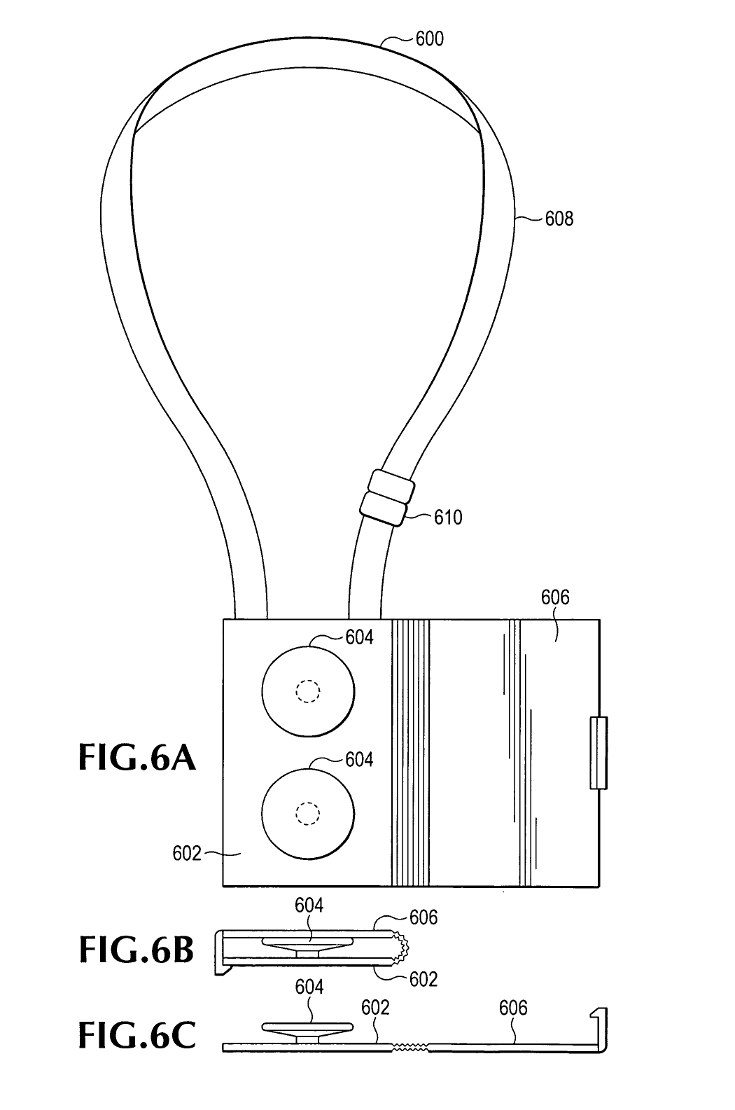

Another alternate embodiment showing use of this system is seen in FIG. 6. In this embodiment, the dressing takes the form of a lanyard 600 worn around the patent's neck. The lanyard has a base member 602 on which are attached two retention wheels 604. A base cover 606 is provided to removably cover the base member 602 and protect a body line secured by at least one retention wheel 604. The lanyard 600 has a neck strap 608 which closes via a safety latch 610 which, in the preferred embodiment, is a break away latch of known invention. This lanyard embodiment may be used with a variety of body lines where only limited dislodgement protection is needed. In the preferred embodiment, the neck strap 608 and base 602 are manufactured of a textile material, while the retention wheels 604 are plastic, and the base cover 606 is of a snap-close variety.

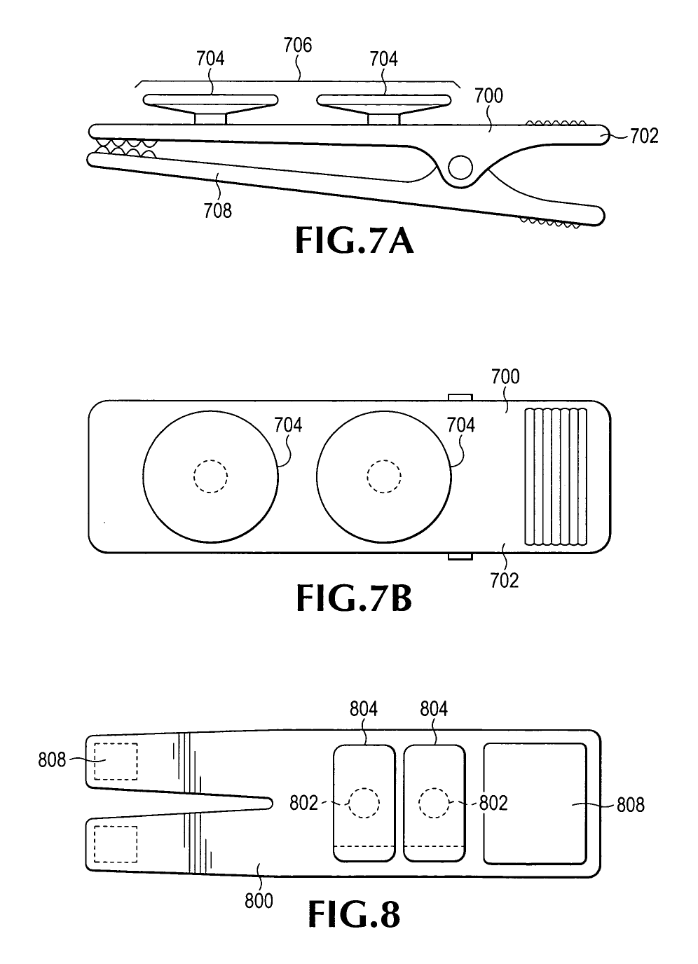

Another alternate embodiment showing use of this system is seen in FIG. 7. As shown, in this embodiment, the dressing is simply an electro line management clip 700. It is well suited for use with a variety of body lines including EKG, blood oxygen monitors, pulse monitors, and even audio ear buds. In this embodiment, the clip 700 has a base member 702 with at least one retention wheel 704, but preferably two wheels 704 arranged in an array 706. The base member 702 is preferably an “alligator clip” of known invention. Alternatively, the base member is affixed to an attachment member 708 which permits the clip 700 to be removably attached to a user's clothing or other dressing. In the preferred embodiment, the clip 700 is reusable and several clips 700 could be used in a series if necessary. The clip 700 may also have a base cover 606 [see FIG. 6] to further aid in securing the body line.

Another alternate embodiment showing use of this system is seen in FIG. 8. As shown, in this embodiment, the dressing takes the form of an arm line cuff 800. When secured to a patient's arm, the cuff 800 is well suited for use with all upper arm lines. The cuff 800 is shown with two retention wheels 802, manufactured of a fabric material similar to the style of the retention wheels 206. Each retention wheel 802 has a cover 804 which is removably secured with Velcro or similar product and which aids in retaining the position of a body line wrapped around the retention wheels 802. The cuff 800 is removably secured to the patient's arm with an arm strap 806 with has an adjustable attachment 808, which in the preferred embodiment is also a Velcro-type attachment.

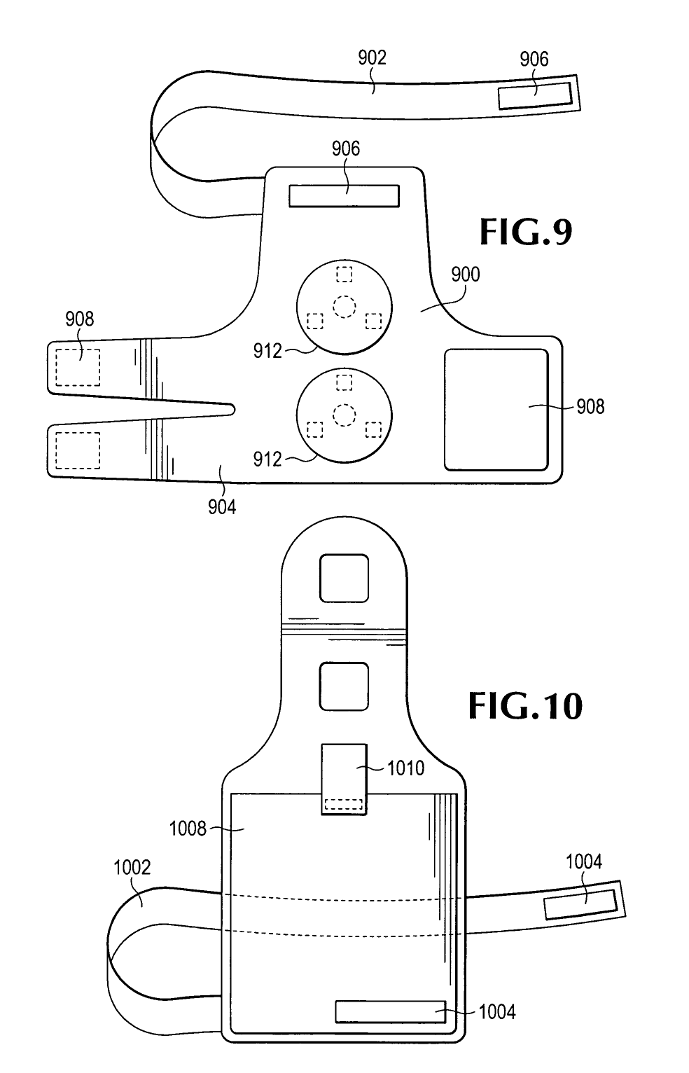

FIG. 9 shows an alternate preferred embodiment. In this embodiment, the dressing take the form a leg line cuff 900 which is secured around a patent's leg with a leg strap 902 and waist strap 904. Each strap 902 and 904 as shown is removably secured with an adjustable attachment 906 and 908 respectively, which is preferably Velcro or similar product. The cuff as shown in FIG. 9 has two retention wheels 912 which are of a style similar to that shown in at 206. The cuff 900 is well suited for most body lines in the lower torso and in the leg.

FIG. 10 shows an alternate embodiment of the leg line cuff 900 wherein the cuff is coupled with a drain bag pouch 1000. The pouch 1000 has a leg strap 1002 which removably attaches the pouch 1000 to a patient's leg with an adjustable attachment 1004. The pouch 1000 fits over a leg line cuff 900 such that retention wheels 912 are inserted through apertures 1006. The pouch has a pocket 1008 into which a drain bag [not shown] could be placed. The pocket 1008 has a closure 1010 to secure the drain bag in the pocket 1008.

The terms and expressions which have been employed in the foregoing specification are used therein as terms of description and not of limitation, and there is no intention, in the use of such terms and expressions, of excluding equivalents of the features shown and described or portions thereof, it being recognized that the scope of the invention is defined and limited only by the claims which follow.

1. A line management system for physically managing a line comprising:
(a) a body dressing;
(b) an array affixed to said body dressing; and
(c) a line which is capable of acting as a conduit and which is closely held to a body by said dressing.

2. The system of claim 1 where the array comprises at least one protrusion with an axis perpendicular to said dressing, where said axis is capable of having a portion of the line wrapped around it.

3. The system of claim 2 said protrusion is a retention wheel.

4. The system of claim 3 where said dressing further comprises an array cover which may be detachably affixed over said array.

5. The system of claim 4 where the body dressing includes an attachment member to closely secure the dressing to a patient.

6. The system of claim 5 where the body dressing is a clip.

7. A method for stabilizing a line, the method comprising the steps of: (a) providing a line which is capable of acting as a conduit; (b) positioning a body dressing, where the body dressing includes at least one array; (c) wrapping a portion of the line around the spatial array on the body dressing; and (d) securing the body dressing so that the line is closely held to a body.

8. The method of claim 7 where said array comprises at least one protrusion with an axis perpendicular to said dressing, where said axis is capable of having at least a portion of the line wrapped around it.

9. The method of claim 8 said protrusion is a retention wheel.

10. The method of claim 8 where said dressing further comprises an array cover which may be detachably affixed over said array.

11. The method of claim 8 where the body dressing includes an attachment member to closely secure the dressing to a patient.

12. The method of claim 11 where the body dressing is a clip.

13. A storage apparatus for body lines comprising:
(a) a body dressing having at least a bottom panel;
(b) at least one wheel for defining a path about which a body line can be wrapped, said wheel having a lower end and an upper end, said wheel having an axis, and said lower end being attached to said bottom panel; and
(c) a guard attached to the upper end of each of said wheels, each guard having a top side and a relatively thin flange that extends from the top side of each guard, and wherein once a body line has been wrapped about said wheels, said guards assist in preventing the body line from unwrapping.

14. The storage apparatus of claim 13, wherein said body dressing is a clip.

15. The storage apparatus of claim 13, where the body dressing includes an attachment member to closely secure the dressing to a patient.

16. The storage apparatus of claim 15, wherein said body dressing further comprises a device storage pocket.

17. The storage apparatus of claim 13, wherein said guard further comprises a wheel cover which may be detachably affixed over said wheel.
